(19) United States
(12) Patent Application Publication
(10) Pub. No.: US 2015/0140674 A1
(43) Pub. Date: May 21, 2015

(54) Method for Determining Weight Concentration of A Polymer Penetrated into A Porous Medium

(71) Applicant: Schlumberger Technology Corporation, Sugar land, TX (US)

(72) Inventors: Dmitry Nikolaevich Mikhailov, Moscow (RU); Evgeny Mikhailovich Chuvilin, Moscow (RU); Lubov Vasilievna Melchakova, Moscow (RU); Tatyana Alexandrovna Buida, Moscow (RU)

(21) Appl. No.: 14/509,016

(22) Filed: Oct. 7, 2014

(30) Foreign Application Priority Data
Nov. 19, 2013 (RU) 2013151154

(51) Int. Cl. G01N 33/24

(52) U.S. Cl. CPC G01N 33/24
USPC 436/94

(57) ABSTRACT

A polymer solution is dried until full evaporation of water. The polymer formed after drying the polymer solution is heated up and active degradation temperature range at a given heating rate and a degree of polymer degradation within this temperature range are determined. Then the solution is dried and a thermal analysis is conducted within the temperature range including the active degradation temperature range. A loss of mass of a portion of a sample of a porous medium is calculated and a loss of mass of similar sample of the porous medium sample after pumping the polymer solution injection is also determined. Based on the derived values, weight concentration of polymer penetrated into the porous medium is determined.

### CROSS-REFERENCE TO RELATED APPLICATION

This application claims priority to Russian Application No. 2013151154 filed Nov. 19, 2013, which is incorporated herein by reference in its entirety.

### BACKGROUND

The invention relates to methods for porous material sample analysis, in particular, it can be used for quantitative analysis of oil and gas formation damage in a near-wellbore zone caused by invasion of polymers contained in drilling muds.

The problem of the near-wellbore formation damage caused by invasion of drilling mud (or circulating fluid) components is especially critical for long horizontal wells because most of them are open-hole completions, i.e. such wells are completed without cemented and perforated production casing.

Drilling muds are complex mixtures of polymers, particles (sized from several millimeters to less than one micron), clays and other additives contained in a “carrier” fluid, which is a “base” of a drilling mud. The base can be either water, oil or a synthetic fluid.

During drilling, mud filtrate as well as fine particles, polymers and other components contained in the mud invade into the near-wellbore zone, resulting in substantial reduction of near-wellbore zone permeability (normally, the terms “damage of near-wellbore formation zone” or simply “formation damage” are used to characterize this phenomenon). In addition, an external filter cake consisting of filtered particles and other components of the drilling mud is deposited on the wellbore wall.

During the well clean-up process (gradual production starting), the exterior filter cake is destroyed, and the invaded components of the drilling mud are partly washed away from the near-wellbore zone and permeability is partly restored. Nevertheless, some mud components remain trapped in the pore space of the rock (by adsorption on pore surface, or seized in narrow pore channels) resulting in a substantial difference between original permeability and permeability restored after the well clean-up procedure (normally, the restored permeability does not exceed 50-70% of the original permeability).

A conventional laboratory method for checking drilling mud quality is a filtration test consisting of mud injection into a core sample and subsequent backflow (i.e. the invaded drilling mud is displaced by the original reservoir fluid). Measurements are made of decreasing and restoring permeability as a function of the amount of fluid (drilling mud or reservoir fluid) injected into pore space.

However, this conventional laboratory method allows measuring an integral hydrodymanic resistance of the core (ratio between current pressure drop across the core and the current rate) only which dynamics is caused by external filter cake behavior and invasion / removal of mud components.

Depth of penetration and concentration of polymers and other mud components entrained in pore space after reverse injection are critical for understanding formation damage mechanisms and selecting an appropriate technique to improve well deliverability (by minimizing near-wellbore formation damage). These parameters cannot be derived from the aforementioned conventional method of evaluating drilling mud quality.

Quantitative analysis of formation damage mechanisms related to invasion of polymers during drilling is of particular interest because polymers (e.g., xanthan gum, carboxymethyl cellulose) are widely used in today's drilling mud systems. The technical problem relates to difficulties of measuring small weight concentration of a polymer in a porous medium.

Patents U.S. Pat. No. 4,540,882 and U.S. Pat. No. 5,0273,79 describe methods for determining drilling mud invasion depth using X-ray computer tomography of a core with a contrast agent addition. But use of a contrast agent dissolved in a “carrier fluid” does not allow evaluation of an invasion depth and concentration of polymers and other low-contrast additives of the drilling mud because invasion depths of mud filtrate and of these additives are generally different.

Patent U.S. Pat. No. 5,253,719 proposes a method for diagnosing formation damage mechanism through the use of radially oriented core samples taken from the well. Core samples are analyzed under a number of analytical methods to determine the type and extent of formation damage and the distance the damage extends out into the formation. X-ray diffraction (XRD) analysis, X-ray micro-analysis, scanning electron microscope (SEM) analysis, backscattered electron microscopy, petrographic analysis, optical microscopy are mentioned among the analytical methods. However, the methods mentioned in the above patent cannot be applied to determine weight concentration of polymers. Besides, polymers have poor contrast to X-rays and, therefore, cannot be distinguished on X-ray computer micro-tomography without special contrast agents.

A widely used method for determining the polymer quantity irreversibly retained in a porous media sample is based on sequential injection of polymer slugs and recording slug front at the exit of the sample by observing changes in polymer concentration in the outgoing fluid (e.g., Zaitoun A., Kohler N., “Two-phase flow though porous media: effect of an adsorbed polymer layer”, SPE 18085, or Zaitoun A., Kohler N., “The role of adsorption in polymer propagation through reservoir rocks”, SPE 16274). On the other hand, the quantity of polymer retained may be evaluated through mass balance of the injected and outgoing polymer.

Disadvantage of these methods is the need to re-inject the polymer slug and to measure concentration of polymer (or a special tracer agent, e.g. Zaitoun A., Kohler N. “The role of adsorption in polymer propagation through reservoir rocks”, SPE 16274) at the exit from the sample. This makes the testing a lengthy process, which requires periodic sampling of the outgoing fluid at the exit or a more sophisticated apparatus.

Another significant disadvantage of the method is that it only provides determination of overall quantity of the polymer retained in the whole porous media sample. This value is only suitable for quantification of permeability reduction mechanism in porous medium that is related to polymer molecule adsorption on pore walls because in this case, if injection volume is large enough (when polymer concentration at the sample exits is constant), the retained polymer would be evenly distributed across the porous medium sample.

However, if size of polymer macromolecules becomes comparable with typical pore size of porous medium (or if microgels are present in the fluid), another polymer retention mechanism is involved: macromolecules become captured by narrow pore channels, e.g. Zitha P. L. J., Chauveteau G., L'eger L. “Unsteady-State Flow of Flexible Polymers in Porous Media”, Journal of Colloid and Interface Science. 2001. Vol. 234, pp. 269-283. In this case, the retained polymer is unevenly distributed in the length of the porous medium sample. The total quantity of polymer irreversibly retained in the entire porous medium sample in this case is not the parameter that can be used for quantification of porous medium permeability reduction mechanism.

### SUMMARY

The disclosure provides for a simple, fast and efficient measurement of weight concentration of a polymer invading in a pore space during injection of polymer fluids without the need to use re-injection of polymer slugs. It results in a much shorter time required for testing and there is no need to measure polymer concentration at the sample exit. In addition, this method can be used to determine a distribution profile of polymer weight concentration along the tested sample.

According to the proposed method, an aqueous polymer solution is prepared and the prepared polymer solution is dried at a temperature not exceeding a temperature of the polymer degradation until full evaporation of water. A polymer formed after drying the polymer solution is heated and a temperature range of active polymer degradation at a given heating rate is determined and also a degree of polymer degradation δ_{pa3JI} within the temperature range of active polymer degradation is determined as

$${δ}_{decomp} = \frac{Δ {M}_{p}}{{M}_{p}^{0}} ,$$

where ΔMₚ—is loss of polymer mass within the temperature range of active polymer degradation, Mₚ⁰—is an initial mass of the polymer before heating.

Then a first sample of the porous medium not containing the polymer is dried at a temperature not exceeding the temperature of the polymer degradation until full evaporation of a pore moisture, and thermal analysis of a portion of the first sample of the porous medium in the temperature range including the temperature range of active polymer degradation at the given heating rate is performed. A loss of mass of the portion of the first sample of the porous medium is calculated when a reference temperature is reached during the thermal analysis, the reference temperature is not less than an upper boundary temperature of the temperature range of active polymer degradation at the given heating rate.

The solution containing the polymer is pumped through a second sample of the porous medium similar to the first sample, and the second sample of the porous medium is dried until full evaporation of pore moisture at the same temperature as the temperature at which the first sample was dried. Thermal analysis of a portion of the second sample of the porous medium is performed at the same heating rate as was used to heat the portion of the first sample and in a temperature range including the temperature range of active polymer degradation at the given heating rate and the reference temperature. A loss of mass of the portion of the second sample of the porous medium when the reference temperature is reached during the thermal analysis is calculated.

The weight concentration of the polymer is calculated as:

$${C}_{p} = \frac{Δ {M}_{second} - Δ {M}_{ref}}{{δ}_{decomp}}$$

where ΔM_{second}—is the loss of mass of the portion of the second sample of the porous medium, ΔM_{ref}—is the loss of mass of the portion of the first sample of the porous medium, δ_{decomp}—is the degree of polymer degradation.

The loss of mass of the portion of the first sample of the porous medium and the loss of mass of the portion of the second sample of the porous medium when the reference temperature is reached, which is not less than an upper boundary temperature of the temperature range of active polymer degradation at the given heating rate can be determined in percent of the original value:

ΔM_{ref}=100*(M⁰_{ref}−M*_{ref})/M⁰_{ref}

where ΔM_{ref}—is the loss of mass of the portion of the first sample of the porous medium, M^{*}_{ref}—a mass of the portion of the first sample of the porous medium at the reference temperature, M⁰_{ref}—an initial mass of the portion of the first sample of the porous medium;

ΔM_{second}=100*(M⁰_{second}−M*_{second})/M⁰_{second}

where ΔM_{second}—is the loss of mass of the portion of the second sample of the porous medium, M*_{second}—a mass of the portion of the second sample of the porous medium at the reference temperature, M⁰_{wlsecond}—an initial mass of the portion of the second sample of the porous medium.

According to one embodiment of the disclosure, a rock core sample is used as the first and the second sample of the porous medium and a drilling mud is used as a fluid containing the polymer.

According to another embodiment, a reservoir fluid is additionally pumped through the second core sample. The reservoir fluid is injected at the opposite end to the end at which the drilling mud was injected.

According to yet another embodiment of the disclosure, after pumping the solution containing the polymer through the second sample of the porous medium, the second sample is split at least in two parts, and a loss of mass of a portion and weight concentration of polymer are determined for each of the split parts. As a result, weight concentration distribution profile along the sample length is determined.

### BRIEF DESCRIPTION OF DRAWINGS

The invention is illustrated by drawings.

FIG. 1 shows thermal curves for the first sample,

FIG. 2 shows thermal curves for the second sample, and

FIG. 3 shows polymer weight concentration distribution profile along the sample.

### DETAILED DESCRIPTION

The physical basis of this method is provided by degradation (decomposition) of a polymer when a certain temperature is reached. For example, xanthan gum begins to degrade at temperature about 250-300° C. Intense degradation of a polymer leads to reduction of sample weight registered by thermal analysis instruments (for example, by derivatograph, thermogravimetric analyzer, etc.). The total weight loss is proportional to mass fraction of the polymer contained in the tested sample.

As an example, measurement of a residual polymer (xanthan gum) was made in Castlegate sandstone sample with 690 mD permeability to water (and 1.2 D permeability to gas) and 25.5% porosity. Thermal analysis was conducted using Q-1500D derivatograph (made in Hungary).

1% water solution of xanthan gum was prepared; NaCl content in solution was 18 g/l. The polymer solution sample was dried at T_{dry}=105° C. until full evaporation of water.

The sample was heated up in Q-1500D derivatograph to determine the temperature range of active degradation of polymer T₁≈220° C., T₂≈400° C., as well as an extent of polymer degradation δ_{decomp}=0.675 at heating rate 20° C./min.

A first sample of Castlegate sandstone (without the polymer) was dried at T_{dry}=105° C. during 24 hours and crushed in a mortar. A portion of the first sample with a mass M⁰_{ref}≈470.2 mg (corresponding to Q-1500D derivarograph characteristics) was taken for thermal analysis (e.g., see Topor N. D., Ogorodova L. P., Melchakova L. V., “Thermal analysis of minerals and inorganic compounds”. Moscow, Moscow State University Publishing House, 1987, p. 6-23.).

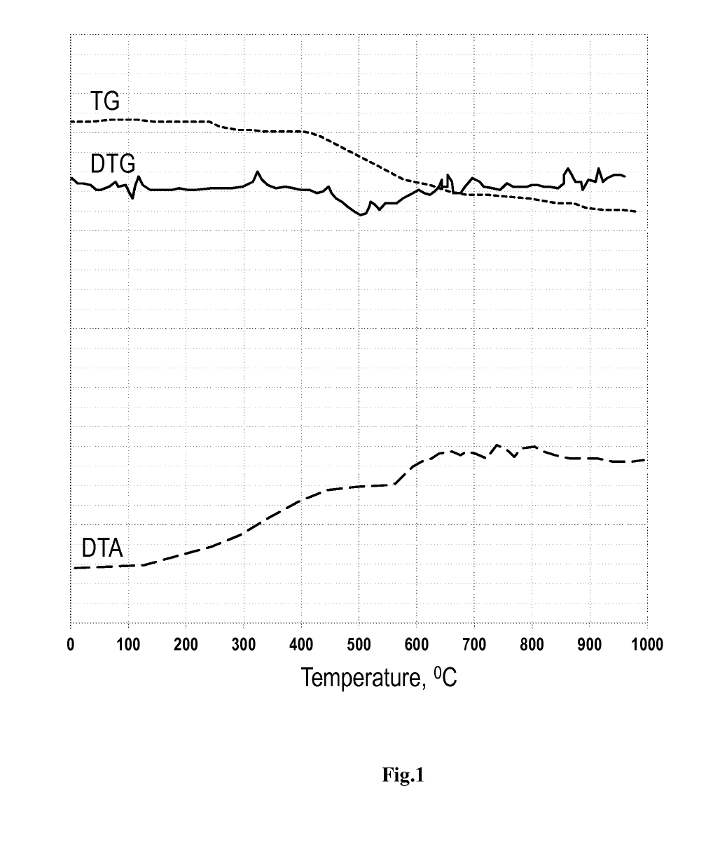

Thermal analysis of the portion was conducted within the temperature range from the room temperature to 1000° C.; thermal curves are shown on FIG. 1. The differential heating curve shows an endothermal effect in the 575° C. area, which corresponds to phase transformation of α-β quartz. The differential thermogravimetric curve and thermogravimetric curve show a loss of mass in the 400-700° C. interval, which is characteristic thermal behavior of some clay minerals, in particular, kaolinite.

Loss of mass of the portion of the first sample was calculated (in percent of the initial value) ΔM_{ref} within the temperature range from 105° to the reference temperature T*=400° C., which corresponds to the upper limit temperature of previously determined active degradation temperature range at a given heating rage, ΔM_{wlref}=0.13%.

This polymer solution (1% xanthan gum water solution with NaCl content 18 g/l) was injected into the second core sample, similar to the first core sample, then NaCl (18 g/l) water solution was injected at the end opposite to the end at which polymer was injected, in order to remove mobile polymer from the sample.

A second sample of Castlegate sandstone was dried at T_{dry}=105° C. during 24 hours and crushed in a mortar. A portion with mass M⁰_{second}=457 mg (corresponding to Q-1500D derivarograph characteristics) was taken from the second sample for thermal analysis

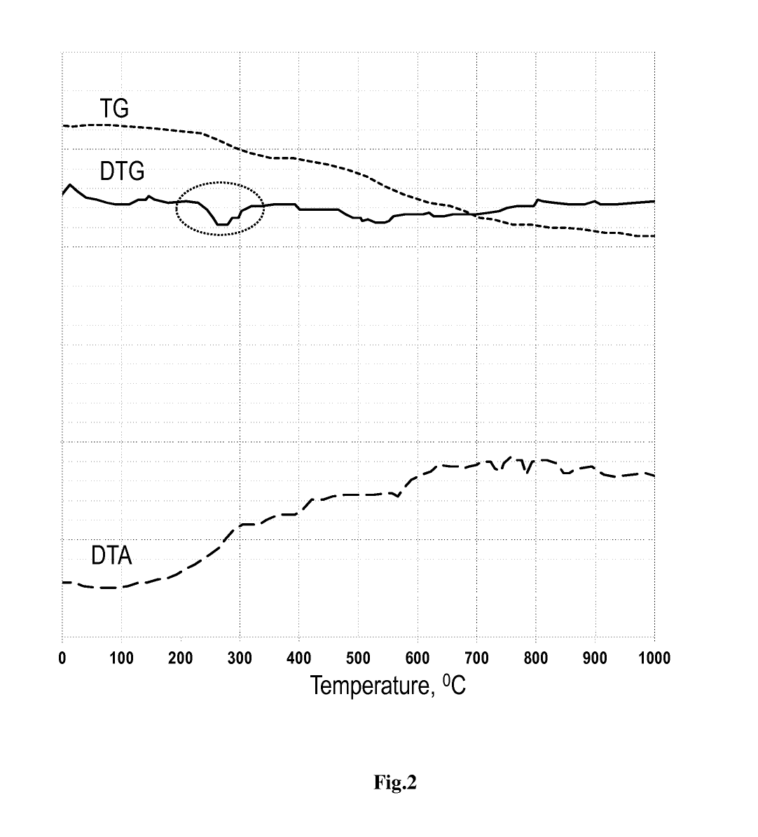

Thermal analysis of the portion of the second sample was conducted within the temperature range from the room temperature to 1000° C., thermal curves are shown on FIG. 2. In addition to the reference sample mass loss described above, mass loss in a lower temperature range (220-400° C.) was recorded here. The differential thermogravimetric curve shows this area as an ellipse, it corresponds to the area of intensive mass loss of the portion of the sample due to polymer degradation.

Mass loss of the portion of the second sample (in percent of the initial value) was ΔM_{second} calculated within the temperature range from 105° to the reference temperature T*=400° C., ΔM_{second}=0.25%

Polymer weight concentration Cₚ (in percent) was calculated:

$${C}_{p} = \frac{Δ {M}_{wl second} - Δ {M}_{wlref}}{{δ}_{decomp}} = \frac{0.25 \% - 0.13 \%}{0.675} \approx 0.18 \%$$

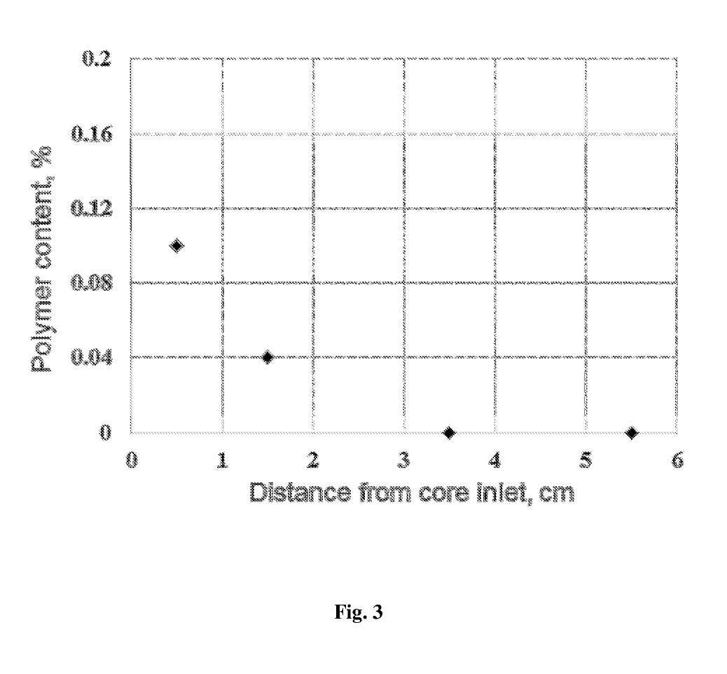

A second example in the early steps is similar to the previous example: a similar Castlegate sandstone sample was used, 1% water solution of xanthan gum with NaCl content of 18 g/l was injected at one end, followed by injection of 18 g/l NaCl solution at the other end. Unlike the first example, the core sample after drilling mud injection was split into 4 parts; further analyses were made separately for each part of the core. As result, a polymer concentration distribution profile was plotted along the core, starting from the core end at which the polymer solution was injected. This profile is shown on FIG. 3.

1. A method for determining weight concentration of a polymer penetrating a porous medium comprising:
preparing an aqueous polymer solution and drying the polymer solution at a temperature not exceeding a temperature of polymer degradation till full evaporation of water,
heating a polymer formed after drying the polymer solution and determining a temperature range of active polymer degradation at a given heating rate and also a degree of polymer degradation δ_{decomp} within the temperature range of active polymer degradation as
$${δ}_{pa 3 JI} = \frac{Δ {M}_{p}}{{M}_{p}^{0}} ,$$
where Δ(M)ₚ—is a loss of polymer mass within the temperature range of active polymer degradation, Mₚ⁰—is an initial mass of the polymer before heating,
drying a first sample of the porous medium not containing the polymer at a temperature not exceeding the temperature of polymer degradation till full evaporation of a pore moisture,
performing thermal analysis of a portion of the first sample of the porous medium in a temperature range including the temperature range of active polymer degradation at the given heating rate,
calculating a loss of mass of the portion of the first sample of the porous medium when a reference temperature is reached during the thermal analysis, the reference temperature is not less than an upper boundary temperature of the temperature range of active polymer degradation at the given heating rate,
pumping the polymer solution through a second sample of the porous medium similar to the first sample and drying the second sample of the porous medium till full evaporation of a pore moisture at the same temperature as the temperature at which the first sample was dried,
performing thermal analysis of a portion of the second sample of the porous medium at the same heating rate as was used to heat the portion of the first sample and in a temperature range including the temperature range of active polymer degradation at the given heating rate and the reference temperature,
calculating a loss of mass of the portion of the second sample of the porous medium when the reference temperature is reached during the thermal analysis, and
calculating the weight concentration of the polymer penetrating the porous medium as
$${C}_{p} = \frac{Δ {M}_{second} - Δ {M}_{ref}}{{δ}_{decomp}}$$
where ΔM_{second}—is the loss of mass of the portion of the second sample of the porous medium, ΔM_{ref}—is the loss of mass of the portion of the first sample of the porous medium, δ_{decomp}—is the degree of polymer degradation.

2. The method of claim 1, wherein the loss of mass of the portion of the first sample of the porous medium and the loss of mass of the portion of the second sample of the porous medium when the reference temperature is reached, are determined in percent of the initial value:
ΔM_{ref}=100*(M⁰_{ref}−M*_{ref})/M⁰_{ref}
where ΔM_{ref}—is the loss of mass of the portion of the first sample of the porous medium, M^{*}_{ref}—a mass of the portion of the first sample of the porous medium at the reference temperature, M⁰_{ref}—an initial mass of the portion of the first sample of the porous medium;
ΔM_{second}=100*(M⁰_{second}−M*_{second})/M⁰_{second}
where ΔM_{second}—is the loss of mass of the portion of the second sample of the porous medium, M*_{second}—a mass of the portion of the second sample of the porous medium at the reference temperature, M⁰_{second}—an initial mass of the portion of the second sample of the porous medium.

3. The method of claim 1, wherein a rock core sample is used as the first and the second porous medium samples and a drilling mud is used as the polymer solution.

4. The method of claim 3, wherein a reservoir fluid is additionally pumped through the core sample, the reservoir fluid is injected at the end opposite to the end at which the drilling mud was injected.

5. The method of claim 1, wherein after pumping the solution through the second sample of the porous medium, the second sample is split at least in two parts, weigh concentration of polymer is determined for each of the split parts and weight concentration distribution profile along the sample length is determined.

6. The method of claim 5, wherein a rock core sample is used as the first and the second samples of the porous medium and a drilling mud is used as a the polymer fluid containing.
